SEQUENCE LISTING (4 sequences)

SEQ ID NO: 1 - DNA, 14 bp, Artificial Sequence
  Oligonucleotide
ccggcccctgaatg

SEQ ID NO: 2 - DNA, 15 bp, Artificial Sequence
  Oligonucleotide
caccggatggccaat

SEQ ID NO: 3 - DNA, 24 bp, Artificial Sequence
  Oligonucleotide
gggcaactctgcagcggaaccgac

SEQ ID NO: 4 - DNA, 20 bp, Artificial Sequence
  Oligonucleotide
tgggtgaccgtgtttctttt

(19) United States
(12) Patent Application Publication
(10) Pub. No.: US 2011/0045458 A1
(43) Pub. Date: Feb. 24, 2011

(54) Detection of Enterovirus

(75) Inventors: Thomas F. Smith, Rochester, MN (US); Mark J. Espy, Rochester, MN (US)

Correspondence Address: FISH & RICHARDSON P.C. (TC), PO BOX 1022, MINNEAPOLIS, MN, 55440-1022, US

(73) Assignee: Mayo Foundation for Medical Education and Research, Rochester, MN (US)

(21) Appl. No.: 12/859,917

(22) Filed: Aug. 20, 2010

Related U.S. Application Data
(60) Provisional application No. 61/235,390, filed on Aug. 20, 2009.

(51) Int. Cl. C12Q 1/70; C07H 21/00

(52) U.S. Cl. 435/5; 536/23.1

(57) ABSTRACT

This document describes methods and materials relating to viral diagnostics, and more particularly to the detection of enterovirus. For example, primers and probes for the detection of enterovirus are provided. Articles of manufacture containing such primers and probes for detecting enterovirus are further provided.

### CROSS-REFERENCE TO RELATED APPLICATIONS

This application claims priority under 35 USC §119(e) to U.S. Patent Application Ser. No. 61/235,390, filed on Aug. 20, 2009, the entire contents of which are hereby incorporated by reference.

### TECHNICAL FIELD

This document provides methods and materials relating to viral diagnostics and, more particularly, to the detection of enterovirus. For example, this document provides methods and materials for detecting enterovirus in cerebrospinal fluid.

### BACKGROUND

The genus Enterovirus consists of more than 60 serologically distinct members within the family Picornaviridae, all of which are known to cause infections in humans. Enteroviruses cause an estimated 10-15 million or more symptomatic infections per year in the United States. The majority of enterovirus infections cause mild or non-symptomatic illness, but some enteroviruses are associated with severe and potentially fatal diseases such as aseptic meningitis, myocarditis, encephalitis, paralysis, and neonatal sepsis. Importantly, the symptoms of enterovirus can be difficult to distinguish from other viral and bacterial febrile illnesses. Therefore, rapid identification of enterovirus infections provides the opportunity to initiate appropriate therapies, and immediate reporting can prevent the unwarranted use of antibiotics.

### SUMMARY

This document describes methods of identifying enterovirus nucleic acid in a biological sample using real-time polymerase chain reaction (PCR). Primers and probes for detecting enterovirus are described herein, as are kits containing such primers and probes. Methods described herein can be used to rapidly identify enterovirus nucleic acid from biological specimens for diagnosis of enterovirus infection. Using specific primers and probes, the methods include amplifying and monitoring the development of specific amplification products using fluorescence resonance energy transfer (FRET).

In general, one aspect of this document features a method for detecting the presence or absence of enterovirus in a biological sample from an individual. The method comprises, or consists essentially of, performing at least one cycling step. The cycling step can comprise an amplifying step and a hybridizing step. The amplifying step can comprise contacting a sample with a pair of enterovirus primers to produce an enterovirus amplification product if an enterovirus nucleic acid molecule is present in the sample. The hybridizing step can comprise contacting the sample with a pair of enterovirus probes. The members of the pair of enterovirus probes can hybridize to the enterovirus amplification product within no more than five nucleotides of each other. A first enterovirus probe of the pair of enterovirus probes can be labeled with a donor fluorescent moiety and the second enterovirus probe of the pair of enterovirus probes can be labeled with a corresponding acceptor fluorescent moiety. The method can comprise detecting the presence or absence of fluorescence resonance energy transfer (FRET) between the donor fluorescent moiety of the first enterovirus probe and the acceptor fluorescent moiety of the second enterovirus probe. The presence of FRET can be indicative of the presence of enterovirus in a sample. The absence of FRET can be indicative of the absence of enterovirus in a sample. The pair of enterovirus primers can comprise a first enterovirus primer and a second enterovirus primer. The first enterovirus primer can comprise the sequence 5′-CCG GCC CCT GAA TG-3′ (SEQ ID NO:1). The second enterovirus primer can comprise the sequence 5′-CAC CGG ATG GCC AAT-3′ (SEQ ID NO:2). The first enterovirus probe can comprise the sequence 5′-GGG CAA CTC TGC AGC GGA ACC GAC-3′ (SEQ ID NO:3). The second enterovirus probe can comprise the sequence 5′-TGG GTG ACC GTG TTT CTT TT-3′ (SEQ ID NO:4). The members of the pair of enterovirus probes can hybridize within no more than two nucleotides of each other. The members of the pair of enterovirus probes can hybridize within no more than one nucleotide of each other. The donor fluorescent moiety can be fluorescein. The corresponding acceptor fluorescent moiety can be selected from the group consisting of LC-Red 640, LC-Red 705, Cy5, and Cy5.5. The detecting step can comprise exciting the sample at a wavelength absorbed by the donor fluorescent moiety and visualizing and/or measuring the wavelength emitted by the corresponding acceptor fluorescent moiety. The detecting can comprise quantitating the FRET. The detecting can comprise quantitating the FRET. The detecting step is performed in real time. The detecting step can be performed after each cycling step. The method can further comprise determining the melting temperature between one or both of the enterovirus probe(s) and the enterovirus amplification product. The presence of the FRET within 45 cycling steps can be indicative of the presence of an enterovirus infection in the individual. The presence of the FRET within 40 cycling steps can be indicative of the presence of an enterovirus infection in the individual. The presence of the FRET within 35 cycling steps can be indicative of the presence of an enterovirus infection in the individual. The method can further comprise preventing amplification of a contaminant nucleic acid. The preventing can comprise performing the amplifying step in the presence of uracil. The preventing can further comprise treating the sample with uracil-DNA glycosylase prior to a first amplifying step. The biological sample can be cerebrospinal fluid. The method cycling step can be performed on a control sample. The control sample can comprise the portion of the enterovirus nucleic acid molecule. The cycling step can use a pair of control primers and a pair of control probes. The control primers and the control probes can be other than the enterovirus primers and enterovirus probes. The amplifying step can produce a control amplification product. The control probes can hybridize to the control amplification product.

In another aspect, this document features an article of manufacture. The article of manufacture can comprise, or consist essentially of, a pair of enterovirus primers; a pair of enterovirus probes; and a donor fluorescent moiety and a corresponding acceptor fluorescent moiety. The pair of enterovirus primers can comprise a first enterovirus primer and a second enterovirus primer. The first enterovirus primer can comprise the sequence 5′-CCG GCC CCT GAA TG-3′ (SEQ ID NO:1). The second enterovirus primer can comprise the sequence 5′-CAC CGG ATG GCC AAT-3′ (SEQ ID NO:2). The pair of enterovirus probes can comprise a first enterovirus probe and a second enterovirus probe. The first enterovirus probe can comprise the sequence 5′-GGG CAA CTC TGC AGC GGA ACC GAC-3′ (SEQ ID NO:3). The second enterovirus probe can comprise the sequence 5′-TGG GTG ACC GTG TTT CTT TT-3′ (SEQ ID NO:4). The first enterovirus probe can be labeled with the donor fluorescent moiety and the second enterovirus probe can be labeled with the corresponding acceptor fluorescent moiety. The article of manufacture can further comprise a package insert having instructions thereon for using the pair of enterovirus primers and the pair of enterovirus probes to detect the presence or absence of enterovirus in a sample.

In another aspect, this document features a method for detecting the presence or absence of enterovirus in a biological sample from an individual. The method can comprise, or consist essentially of, performing at least one cycling step. The cycling step can comprise an amplifying step and a hybridizing step. The amplifying step can comprise contacting said sample with a pair of enterovirus primers to produce an enterovirus amplification product if an enterovirus nucleic acid molecule is present in the sample. The hybridizing step can comprise contacting the sample with an enterovirus probe. The enterovirus probe can be labeled with a donor fluorescent moiety and a corresponding acceptor fluorescent moiety. The method can comprise detecting the presence or absence of fluorescence resonance energy transfer (FRET) between the donor fluorescent moiety and the acceptor fluorescent moiety of the enterovirus probe. The presence or absence of fluorescence can be indicative of the presence or absence of enterovirus in the sample. The amplification can employ a polymerase enzyme having 5′ to 3′ exonuclease activity. The first and second fluorescent moieties can be within no more than 5 nucleotides of each other on the probe. The second fluorescent moiety can be a quencher. The enterovirus probe can comprise a nucleic acid sequence that permits secondary structure formation. The secondary structure formation can result in spatial proximity between the first and second fluorescent moiety. The second fluorescent moiety can be a quencher.

In another method, this document also features a method for detecting the presence or absence of enterovirus in a biological sample from an individual. The method can comprise, or consist essentially of, performing at least one cycling step. A cycling step can comprise an amplifying step and a dye-binding step. The amplifying step can comprise contacting the sample with a pair of enterovirus primers to produce an enterovirus amplification product if an enterovirus nucleic acid molecule is present in the sample. The dye-binding step can comprise contacting the enterovirus amplification product with a double-stranded DNA binding dye. The method can comprise detecting the presence or absence of binding of the double-stranded DNA binding dye into the amplification product. The presence of binding can be indicative of the presence of enterovirus in the sample. The absence of binding can be indicative of the absence of enterovirus in the sample. The double-stranded DNA binding dye can be ethidium bromide. The method can further comprise determining the melting temperature between the enterovirus amplification product and the double-stranded DNA binding dye. The melting temperature can confirm the presence or absence of the enterovirus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of this document are set forth in the accompanying drawings and the description below. Other features, objects, and advantages of this document will be apparent from the drawings and detailed description, and from the claims.

### DETAILED DESCRIPTION

A real-time assay for detecting enterovirus in a biological sample is described herein. The real-time assay described herein is more sensitive and specific than existing assays. For example, primers and probes for detecting enterovirus infections are provided as are articles of manufacture containing such primers and probes. The increased sensitivity of real-time PCR for detection of enterovirus compared to other methods, as well as the improved features of real-time PCR including sample containment and real-time detection of the amplified product, make feasible the implementation of this technology for routine diagnosis of enterovirus infections in the clinical laboratory.

The methods and materials described herein can be used to detect and amplify enterovirus genomes of the poliovirus, coxsackievirus, and echovirus groups. For example, the methods and materials described herein can be used to detect the following enteroviruses: coxsackie A strains 2, 3, 5, 7, 9, 10, 11, 12, 13, 14, 15, 17, 18, 19, 20, and 22; coxsackie B strains 1, 2, 3, 4, and 6; poliovirus strains 1, 2, and 3; and echovirus strains 1, 2, 3, 4, 5, 6, 7, 8, 9, 11, 12, 13, 14, 15, 16, 17, 18, 19, 20, 21, 22, 24, 25, 26, 27, 29, 30, 32, 33, and 71. The methods and materials described herein likely can be used to detect the following enteroviruses: coxsackie A strains 1, 4, 6, 8, 16, and 21; coxsackie B strain 5; and echovirus strains 69 and 70; while the methods and materials described herein likely cannot be used to detect the following enteroviruses: echovirus strains 31 and 68.

### Enterovirus Nucleic Acids and Oligonucleotides

This document describes methods and materials that can be used to detect enteroviruses by amplifying, for example, a portion of an enterovirus nucleic acid. Enteroviruses and other members of the Picornaviridae family are small, icosahedral, non-enveloped viruses with a single positive-strand RNA genome. Enterovirus genomic RNA is approximately 7500 nucleotides in length and encodes a large polypeptide. The polypeptide-encoding region is preceded by a long 5′ untranslated region (5′ UTR) of approximately 750 nucleotides followed by a shorter 3′UTR of approximately 70-100 nucleotides and a poly(A) tail. See Hyypia et al., “Classification of Enteroviruses Based on Molecular and Biological Properties,” J. Gen. Virol., 78:1-11 (1997). Enterovirus nucleic acid sequences are available. See, for example, the Virus genome database at NCBI Entrez Genomes (ncbi.nlm.nih.gov/sites/entrez?db=Genome on the World Wide Web) and the Picornavirus sequence database (picornaviridae.com/sequences/sequences.htm on the World Wide Web).

Specifically, primers and probes are provided herein that can be used to amplify and detect enterovirus nucleic acid molecules. Enterovirus nucleic acids other than those exemplified herein also can be used to detect enterovirus in a sample. Enterovirus nucleic acids other than those exemplified herein (e.g., functional variants) can be evaluated (e.g., for specificity and/or sensitivity) by those of skill in the art using routine methods such as, but not limited to, the methods exemplified herein. Representative functional variants, for example, include deletions of, insertions in, and/or substitutions in the nucleic acids disclosed herein.

Primers that amplify an enterovirus nucleic acid molecule (e.g., the 5′ UTR of an enterovirus genome) can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 (e.g., 16, 18, 20, 21, 22, 23, 24, or 25) nucleotides in length.

Designing oligonucleotides to be used as hybridization probes can be performed in a manner similar to the design of primers, although the members of a pair of probes preferably anneal to an amplification product within no more than 5 nucleotides of each other on the same strand such that FRET can occur (e.g., within no more than 1, 2, 3, or 4 nucleotides of each other). This minimal degree of separation typically brings the respective fluorescent moieties into sufficient proximity such that FRET occurs. It is to be understood, however, that other separation distances (e.g., 6 or more nucleotides) are possible provided the fluorescent moieties are appropriately positioned relative to each other (for example, with a linker arm) such that FRET can occur. In addition, probes can be designed to hybridize to targets that contain a polymorphism or mutation, thereby allowing differential detection of enterovirus strains based on either absolute hybridization of different pairs of probes corresponding to the particular enterovirus strain to be distinguished or differential melting temperatures between, for example, members of a pair of probes and each amplification product corresponding to an enterovirus strain to be distinguished. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

As used herein, constructs include vectors containing an enterovirus nucleic acid molecule (e.g., SEQ ID NOs:1, 2, 3, or 4). Constructs can be used, for example, as control template nucleic acid molecules. Constructs or vectors suitable for use in the methods described herein are commercially available and/or produced by recombinant nucleic acid technology methods routine in the art. Enterovirus nucleic acid molecules can be obtained, for example, by chemical synthesis, direct cloning from enterovirus, or by PCR amplification. An enterovirus nucleic acid molecule or fragment thereof can be operably linked to a promoter or other regulatory element such as an enhancer sequence, a response element, or an inducible element that modulates expression of the enterovirus nucleic acid molecule. As used herein, operably linking refers to connecting a promoter and/or other regulatory elements to an enterovirus nucleic acid molecule in such a way as to permit and/or regulate expression of the enterovirus nucleic acid molecule. A promoter that does not normally direct expression of enterovirus can be used to direct transcription of an enterovirus nucleic acid using, for example, a viral polymerase, a bacterial polymerase, or a eukaryotic RNA polymerase II. Alternatively, an enterovirus native promoter can be used to direct transcription of an enterovirus nucleic acid. In addition, operably linked can refer to an appropriate connection between an enterovirus promoter or regulatory element and a heterologous coding sequence (i.e., a non-enterovirus coding sequence, for example, a reporter gene) in such a way as to permit expression of the heterologous coding sequence.

Constructs suitable for use in the methods described herein typically include, in addition to enterovirus nucleic acid molecules (e.g., a nucleic acid molecule that contains one or more sequences of SEQ ID NOs:1, 2, 3, or 4), sequences encoding a selectable marker (e.g., an antibiotic resistance gene) for selecting desired constructs and/or transformants, and an origin of replication. The choice of construct or vector systems usually depends upon several factors, including, but not limited to, the choice of host cells, replication efficiency, selectability, inducibility, and the ease of recovery.

Constructs containing enterovirus nucleic acid molecules can be propagated in a host cell using methods well known in the art. As used herein, the term host cell is meant to include prokaryotes and eukaryotes such as yeast, plant and animal cells. Prokaryotic hosts may include E. coli, Salmonella typhimurium, Serratia marcescens and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct or vector can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Polymerase Chain Reaction (PCR)

U.S. Pat. Nos. 4,683,202, 4,683,195, 4,800,159, and 4,965,188 disclose conventional polymerase chain reaction (PCR) techniques. PCR typically employs two oligonucleotide primers that bind to a selected nucleic acid template (e.g., DNA or RNA). Primers useful for the methods provided herein include oligonucleotides capable of acting as a point of initiation of nucleic acid synthesis within enterovirus nucleic acid sequences (e.g., SEQ ID NO:1, 2, 3, or 4). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T aquaticus, T lacteus, T. rubens, Bacillus stearothermophilus, and Methanothermus fervidus. Nonetheless, polymerases that are not thermostable also can be employed in PCR assays provided the enzyme is replenished.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 seconds to 4 minutes (e.g., 1 minute to 2 minutes 30 seconds, or 1.5 minutes).

If the double-stranded template nucleic acid is denatured by heat, the reaction mixture is allowed to cool to a temperature that promotes annealing of each primer to its target sequence on the enterovirus nucleic acid. The temperature for annealing is usually from about 35° C. to about 65° C. (e.g., about 40° C. to about 60° C.; about 45° C. to about 50° C.). Annealing times can be from about 10 seconds to about 1 minute (e.g., about 20 seconds to about 50 seconds; about 30 seconds to about 40 seconds). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° to 80° C. (e.g., about 50° C. to about 70° C.; about 60° C.). Extension times can be from about 10 seconds to about 5 minutes (e.g., about 30 seconds to about 4 minutes; about 1 minute to about 3 minutes; about 1 minute 30 seconds to about 2 minutes).

PCR assays can employ enterovirus nucleic acid such as RNA or DNA (cDNA). The template nucleic acid need not be purified; it may be a minor fraction of a complex mixture, such as enterovirus nucleic acid contained in human cells or in a biological sample. Enterovirus nucleic acids may be extracted from a biological sample by routine techniques such as those described in Diagnostic Molecular Microbiology: Principles and Applications (Persing et al. (eds), 1993, American Society for Microbiology, Washington D.C) or U.S. Pat. No. 6,811,971. Nucleic acids can be obtained from any number of sources, such as plasmids, or natural sources including bacteria, yeast, viruses, organelles, or higher organisms such as plants or animals.

The oligonucleotide primers (e.g., SEQ ID NO:1 or 2) can be combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO. The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target enterovirus nucleic acid molecule. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps typically will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor and a corresponding acceptor fluorescent moiety are positioned within a certain distance of each other, energy transfer takes place between the two fluorescent moieties that can be visualized or otherwise detected and/or quantitated. Two oligonucleotide probes (e.g., SEQ ID NO:3 or 4), each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. (e.g., about 40° C. to about 60° C.; about 45° C. to about 55° C.; about 50° C.) for about 10 seconds to about 1 minute (e.g., about 20 seconds to about 50 seconds; about 30 seconds to about 40 seconds).

Fluorescent analysis can be carried out using, for example, a photon counting epifluorescent microscope system (containing the appropriate dichroic mirror and filters for monitoring fluorescent emission at the particular range), a photon counting photomultiplier system, or a fluorometer. Excitation to initiate energy transfer can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimidyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and corresponding acceptor fluorescent moieties. The length of a linker arm for the purpose of the methods provided herein is the distance in Angstroms (Å) from the nucleotide base to the fluorescent moiety. In general, a linker arm is from about 10 to about 25 Å (e.g., about 15 Å to about 20 Å). The linker arm may be of the kind described in WO 84/03285. WO 84/03285 also discloses methods for attaching linker arms to a particular nucleotide base, and also for attaching fluorescent moieties to a linker arm.

An acceptor fluorescent moiety such as an LCT™-Red 640-NHS-ester can be combined with C6-Phosphoramidites (available from ABI (Foster City, Calif.) or Glen Research (Sterling, Va.)) to produce, for example, LC™-Red 640-Phosphoramidite. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

### Detection of Enterovirus

Traditionally, cell and tissue culture methods have been used for diagnosis of enterovirus infections. For example, tube cell cultures of human fibroblasts and primary monkey kidney cells inoculated with biological samples from a patient have been used to detect the presence of enterovirus infections. Such assays depend on the recognition of cytopathic effect (CPE) and/or hemadsorption for viral detection. Unfortunately, some enteroviruses grow poorly in the cell types typically used for cell culture assays.

As a substitute for conventional tube cell cultures of human fibroblasts or primary monkey kidney cells, rapid shell vial cell culture assays have been developed that are more efficient and reduce the culture time for virus detection from several days to one or two days after receipt of the specimen into the laboratory. While rapid shell vial assays allow earlier detection and identification of enteroviruses, they are reportedly markedly less sensitive than conventional enterovirus isolation procedures. See, e.g., Van Doornum & De Jong, “Rapid Shell Vial Culture Technique for Detection of Enteroviruses and Adenoviruses in Fecal Specimens: Comparison with Conventional Virus Isolation Method”, J. Clin. Microbiol. 36(10):2865-2868 (1998).

In some cases, direct immunofluorescence assays (DFA) can be used to detect enterovirus infected cells. DFA involves the addition of fluorescently-labeled antibodies to detect the virus of interest in a patient specimen. The sensitivity of this labor-intensive DFA procedure, however, can be as low as 67% compared to cell culture methods such as those described above. In addition, the quality and interpretation of results can be difficult, subjective, and dependent on the quality of the specimens and microscopic equipment. In routine virology practice, DFA is not a stand-alone diagnostic technique for the diagnosis of enterovirus infections.

PCR methods can be used to detect the presence or absence of enterovirus nucleic acids. For example, enterovirus oligonucleotides can be used to detect enteroviruses through PCR amplification and hybridization (e.g., Southern blot hybridization). See Chapman et al., “Molecular Detection and Identification of Enteroviruses Using Enzymatic Amplification and Nucleic Acid Hybridization”, J. Clin. Microbiol. 28(5): 843-850 (1990). In some cases, enterovirus detection can be performed according to the PCR methods described by Rotbart et al. in U.S. Pat. No. 5,075,212. For example, enterovirus oligonucleotides can be used to detect enteroviruses through real-time PCR with radiolabeled probes and slot-blot hybridization.

By using commercially-available real-time PCR instrumentation (e.g., LIGHTCYCLER™, Roche Molecular Biochemicals, Indianapolis, Ind.), PCR amplification and detection of the amplification product can be combined in a single closed cuvette with dramatically reduced cycling time. Since detection occurs concurrently with amplification, the real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory.

This document describes methods for detecting the presence or absence of enterovirus in a biological sample from an individual. Methods described herein avoid problems of sample contamination, false negatives, and false positives. The methods provided herein can be used to determine whether or not a patient is infected with enterovirus or is in need of treatment for a different microorganism. For example, a sample of cerebrospinal fluid (CSF) obtained from an individual who exhibits signs and symptoms consistent with meningitis or encephalitis can be assayed according to the methods provided herein to detect the presence or absence of enterovirus. If positive, the patient can be treated with any appropriate therapeutic regimen and/or administered an appropriate medication (e.g., an antiviral medication) in a timely manner. A positive result also can rule out the possibility of more serious infections. If negative, the patient can be subjected to further screening and/or treatment with appropriate antiviral or antibiotic medications. Prompt screening of individuals for enterovirus also can be used to identify carriers of the virus, which in turn can be used to reduce or eliminate the transmission of virus from carriers to non-carriers.

The methods described herein can be used to detect a number of strains of enteroviruses. For example, the methods can be used to detect the following strains: coxsackie A strains 2, 3, 5, 7, 9, 10, 11, 12, 13, 14, 15, 17, 18, 19, 20, and 22; coxsackie B strains 1, 2, 3, 4, and 6; poliovirus strains 1, 2, and 3; and echovirus strains 1, 2, 3, 4, 5, 6, 7, 8, 9, 11, 12, 13, 14, 15, 16, 17, 18, 19, 20, 21, 22, 24, 25, 26, 27, 29, 30, 32, 33, and 71. The methods and materials described herein likely can be used to detect the following enteroviruses: coxsackie A strains 1, 4, 6, 8, 16, and 21; coxsackie B strain 5; and echovirus strains 69 and 70; while the methods and materials described herein likely cannot be used to detect the following enteroviruses: echovirus strains 31 and 68.

The methods described herein include performing at least one cycling step, which includes amplifying a portion of an enterovirus nucleic acid molecule from a biological sample using a pair of enterovirus primers. Each member of the pair of enterovirus primers anneals to a target within or adjacent to an enterovirus nucleic acid molecule such that at least a portion of the resulting amplification product contains nucleic acid sequence corresponding to enterovirus. More importantly, the amplification product should contain the nucleic acid sequences that are complementary to the enterovirus probes. An enterovirus amplification product is produced when enterovirus nucleic acid is present. Each cycling step further includes contacting the sample with a pair of enterovirus probes. As described herein, one member of each pair of the enterovirus probes typically is labeled with a donor fluorescent moiety while the other is labeled with a corresponding acceptor fluorescent moiety. The presence or absence of FRET between the donor fluorescent moiety of the first enterovirus probe and the corresponding acceptor fluorescent moiety of the second enterovirus probe is detected upon hybridization of the enterovirus probes to the enterovirus amplification product. Each cycling step includes an amplification step and a hybridization step, and each cycling step is usually followed by a FRET detecting step. Multiple cycling steps are performed, preferably in a thermocycler. Detection of FRET in the enterovirus reaction indicates the presence of an enterovirus.

As used herein, “amplifying” refers to the process of synthesizing nucleic acid molecules that are complementary to one or both strands of a template nucleic acid molecule (e.g., enterovirus nucleic acid). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., PLATINUM® Taq) and an appropriate buffer and/or co-factors for optimal activity of the polymerase enzyme (e.g., MgCl₂ and/or KCl).

If amplification of enterovirus nucleic acid occurs and an amplification product is produced, the step of hybridizing results in a detectable signal based upon FRET between the members of the pair of probes. As used herein, “hybridizing” refers to the annealing of probes to an amplification product. Hybridization conditions typically include a temperature that is below the melting temperature of the probes but that avoids non-specific hybridization of the probes.

Generally, the presence of FRET indicates the presence of enterovirus in the sample, and the absence of FRET indicates the absence of enterovirus in the sample. Inadequate specimen collection, transportation delays, inappropriate transportation conditions, or use of certain collection swabs (e.g., calcium alginate swabs or aluminum shaft swabs) are all conditions that can affect the success and/or accuracy of a test result, however. Using the methods disclosed herein, detection of FRET within 45 cycling steps is indicative of an enterovirus infection.

Methods described herein also can be used for enterovirus vaccine efficacy studies or epidemiology studies. For example, an enterovirus vaccine can be detected in a biological sample using the methods described herein during the time when virus is still present in an individual. For such vaccine efficacy studies, the methods described herein can be used to determine, for example, the persistence of an attenuated strain of enterovirus used in a vaccine, or can be performed in conjunction with an additional assay such as a serologic assay to monitor an individual's immune response to such a vaccine. In addition, methods of this document can be used to distinguish one enterovirus strain from another for epidemiology studies of, for example, the origin or severity of an outbreak of enterovirus.

Representative biological samples that can be used in practicing the methods described herein include, without limitation, CSF, nasopharyngeal swabs, throat washings, specimens from bronchial lavage, rectal swabs, and stool samples. Collection and storage methods of biological samples are known to those of skill in the art. Biological samples can be processed (e.g., by nucleic acid extraction methods and/or kits known in the art) to release enterovirus nucleic acid, or in some cases, the biological sample can be contacted directly with the PCR reaction components and the appropriate oligonucleotides. For example, nucleic acids can be extracted from biological samples using the commercially-available total nucleic acid kit, MagNA Pure (Roche Applied Science, Indianapolis, Ind.).

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the enterovirus probes from the enterovirus amplification product can confirm the presence or absence of enterovirus in the sample. For example, the presence of enterovirus in a sample can be confirmed by detecting a melting curve within an established temperature range. In some cases, a melting curve positive for the presence of enterovirus nucleic acid can be detected in the range of about 53° C.±about 7° C. (e.g., about 46, 47, 48, 49, 50, 51, 52, 53, 54, 55, 56, 57, 58, 59, or 60° C.).

Within each thermocycler run, control samples are cycled as well. Positive control samples can amplify enterovirus nucleic acid control template (e.g., other than enterovirus) using, for example, control primers and control probes. Positive control samples can also amplify, for example, a plasmid construct containing enterovirus nucleic acid molecules. Such a plasmid control can be amplified internally (e.g., within the sample) or in a separate sample run side-by-side with the patients' samples. Each thermocycler run should also include a negative control that, for example, lacks enterovirus template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

In an embodiment, the methods disclosed herein include steps to avoid contamination. For example, an enzymatic method utilizing uracil-DNA glycosylase is described in U.S. Pat. Nos. 5,035,996; 5,683,896; and 5,945,313 to reduce or eliminate contamination between one thermocycler run and the next. In addition, standard laboratory containment practices and procedures are desirable when performing methods of this document. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

Conventional PCR methods in conjunction with FRET technology can be used to practice the methods described herein. In one embodiment, a LIGHTCYCLER™ instrument is used. A detailed description of the LIGHTCYCLER™ System and real-time and on-line monitoring of PCR can be found at biochem.roche.com/lightcycler on the World Wide Web. The following patent applications describe real-time PCR as used in the LIGHTCYCLER™ technology: WO 97/46707, WO 97/46714 and WO 97/46712. The LIGHTCYCLER™ instrument is a rapid thermal cycler combined with a microvolume fluorometer utilizing high quality optics. This rapid thermocycling technique uses thin glass cuvettes as reaction vessels. Heating and cooling of the reaction chamber are controlled by alternating heated and ambient air. Due to the low mass of air and the high ratio of surface area to volume of the cuvettes, very rapid temperature exchange rates can be achieved within the LIGHTCYCLER™ thermal chamber. Addition of selected fluorescent dyes to the reaction components allows the PCR to be monitored in real time and on-line. Furthermore, the cuvettes serve as an optical element for signal collection (similar to glass fiber optics), concentrating the signal at the tip of the cuvette. The effect is efficient illumination and fluorescent monitoring of microvolume samples.

The LIGHTCYCLER™ carousel that houses the cuvettes can be removed from the instrument. Therefore, samples can be loaded outside of the instrument (in a PCR Clean Room, for example). In addition, this feature allows for the sample carousel to be easily cleaned and sterilized. The fluorometer, as part of the LIGHTCYCLER™ apparatus, houses the light source. The emitted light is filtered and focused by an epi-illumination lens onto the top of the cuvette. Fluorescent light emitted from the sample is then focused by the same lens, passed through a dichroic mirror, filtered appropriately, and focused onto data-collecting photohybrids. The optical unit currently available in the LIGHTCYCLER™ instrument (Roche Molecular Biochemicals, Catalog No. 2 011 468) includes three band-pass filters (530 nm, 640 nm, and 710 nm), providing three-color detection and several fluorescence acquisition options. Data collection options include once per cycling step monitoring, fully continuous single-sample acquisition for melting curve analysis, continuous sampling (in which sampling frequency is dependent on sample number) and/or stepwise measurement of all samples after defined temperature interval.

The LIGHTCYCLER™ can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As described herein, amplification products can be detected using labeled hybridization probes that take advantage of FRET technology. A common format of FRET technology utilizes two hybridization probes. Each probe can be labeled with a different fluorescent moiety and are generally designed to hybridize in close proximity to each other (e.g., within 5 nucleotides) in a target DNA molecule (e.g., an amplification product). A donor fluorescent moiety, for example, fluorescein, is excited at 470 nm by the light source of the LIGHTCYCLER™ Instrument. During FRET, the fluorescein transfers its energy to an acceptor fluorescent moiety such as LIGHTCYCLER™-Red 640 (LC™-Red 640) or LIGHTCYCLER™-Red 705 (LC™-Red 705). The acceptor fluorescent moiety then emits light of a longer wavelength, which is detected by the optical detection system of the LIGHTCYCLER™ instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target nucleic acid molecules (e.g., the number of enterovirus genomes).

Another FRET format utilizes TAQMAN® technology to detect the presence or absence of an amplification product, and hence, the presence or absence of enterovirus. TAQMAN® technology utilizes one single-stranded hybridization probe labeled with two fluorescent moieties. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target nucleic acid (i.e., the amplification product) and is degraded by the 5′ to 3′ exonuclease activity of the Taq Polymerase during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems, Foster City, Calif.) uses TAQMAN® technology, and is suitable for performing the methods described herein for detecting enterovirus. Information on PCR amplification and detection using an ABI PRISM® 7700 system can be found at appliedbiosystems.com/products on the World Wide Web.

Molecular beacons in conjunction with FRET also can be used to detect the presence of an amplification product using the real-time PCR methods of this document. Molecular beacon technology uses a single hybridization probe labeled at each end of the probe with a first fluorescent moiety and a second fluorescent moiety. Oftentimes, the second fluorescent moiety is a quencher. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

As an alternative to FRET, an amplification product can be detected using a double-stranded DNA binding dye such as a fluorescent DNA binding dye (e.g., SYBRGREEN I® or SYBRGOLD® (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

It is understood that the present invention is not limited by the configuration of one or more commercially available instruments.

### Articles of Manufacture/Kits

This document further describes articles of manufacture that can be used to detect enterovirus. An article of manufacture as described herein can include primers and probes used to detect enterovirus, together with suitable packaging materials. Representative primers and probes for detection of enterovirus are capable of hybridizing to enterovirus nucleic acid molecules. Methods of designing primers and probes are disclosed herein, and representative examples of primers and probes that amplify and hybridize to enterovirus nucleic acid molecules are provided (e.g., SEQ ID NOs: 1, 2, 3 and 4).

Articles of manufacture as described herein also can include one or more fluorescent moieties for labeling the probes or, alternatively, the probes supplied with the kit can be labeled. For example, an article of manufacture may include a donor fluorescent moiety for labeling one of the enterovirus probes and an acceptor fluorescent moiety for labeling the other enterovirus probe, respectively. Examples of suitable FRET donor fluorescent moieties and corresponding acceptor fluorescent moieties are provided above.

Articles of manufacture also can contain a package insert or package label having instructions thereon for using the primers and probes to detect enterovirus in a biological sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

This document will be further described in the following examples, which do not limit the scope of this document described in the claims. The skilled person will understand that various additional features described above may be integrated into the methods and/or articles of manufacture/kits either together or separately.

### EXAMPLES

### Example 1

### Nucleic Acid Extraction for RT-PCR

Nucleic acid extracted from patient biological samples was tested for the presence of enterovirus RNA. Viral nucleic acid was extracted from cerebrospinal fluid (CSF) using either automated or manual extraction. RNA was extracted from 200 μL of patient specimen using the automated MagNA Pure Nucleic Acid extraction kit (Roche Applied Science, Indianapolis, Ind.) and the MagNA Pure Blood/Serum/Plasma extraction program. In addition, nucleic acids were extracted from an enterovirus culture for use as a positive control and from buffer containing 1×10³ E. coli for use as a negative control. The samples were eluted in 100 μL. As an alternative to automated nucleic acid extraction, RNA was manually extracted from 200 μL of patient specimen using HighPure (Roche) and eluted with 100 μL of elution buffer.

Viral RNA was reverse transcribed and amplified using the LightCycler® RNA Master HybProbe (Roche Diagnostics Indianapolis, Ind.). For each reaction, 5 μL of nucleic acid extracted from the biological sample was added to 15 μL of Master Mix. Table 1 shows the contents of the Master Mix, which includes both primer sequences (Primer 1: 5′-CCG GCC CCT GAA TG-3′ (SEQ ID NO:1); Primer 2: 5′-CAC CGG ATG GCC AAT-3′ (SEQ ID NO:2)) and both probe sequences (Probe-FL: 5′-GGG CAA CTC TGC AGC GGA ACC GAC-3′ (SEQ ID NO:3); Probe-Red: 5′-TGG GTG ACC GTG TTT CTT TT-3′ (SEQ ID NO:4)).

Table 2 shows the conditions used to reverse-transcribe, amplify, and detect enterovirus nucleic acid. Briefly, each sample of nucleic acid+Master Mix was incubated at 61° C. for 20 minutes to reverse transcribe viral RNA into cDNA. The samples were then heated at 95° C. for 2-3 minutes to denature the cDNA/RNA hybrid, and real-time PCR and subsequent melting curve analysis was performed on the cDNA as indicated in Table 2.

TABLE 1
Real-Time PCR Master Mix

| | Concentration of stock | μL of stock | Final |
|---|---|---|---|
| Ingredient | solution | solution | concentration |
| Water | — | 188 | — |
| Mn ac^{a} | 50 mM | 42 | 3 mM |
| Buffer^{b} | 2.7x | 259.25926 | 1x |
| Primer 1 | 50 μM | 4.9 | 0.35 μM |
| Primer 2 | 50 μM | 9.8 | 0.7 μM |
| Probe-Fluorescein | 20 μM | 7 | 0.2 μM |
| Probe-RED 640 | 20 μM | 14 | 0.4 μM |

^{a}manganese acetate;
^{b}MagNA Pure buffer or HighPure buffer

TABLE 2
Real-Time PCR Conditions

| | | | Hold time | T transition | Acquisition |
|---|---|---|---|---|---|
| Program | Cycles | ° C. | (mm:ss) | (° C./sec) | Mode |
| Initial | 1 | 61 | 20:00 | 20 | |
| | | 95 | 3:00 | 20 | |
| PCR | 45 | 95 | 0:10 | 20 | |
| | | 52 | 0:15 | 20 | Single |
| | | 72 | 0:15 | 20 | |
| Melting Curve | 1 | 95 | 0:00 | 20 | |
| | | 55 | 0:20 | 20 | |
| | | 40 | 0:00 | 0.2 | |
| | | 85 | 0:00 | 0.2 | Continuous |
| Cool | 1 | 40 | 0:00 | 20 | |

The amplification product was monitored for the development of target nucleic acid sequences after the annealing step during real-time PCR cycling (LIGHTCYCLER®, Roche) using fluorescence resonance energy transfer technology (FRET). Analysis of the real-time PCR amplification and probe melting curves was by LIGHTCYCLER® software. The reaction yielded a “positive” result if a melting curve was present within ±2° C. of a positive control sample falling within the established range of 53° C.±7° C., and if the signal was at least 2 times greater than the signal observed for known negative samples.

### Example 2

### Cell Cultures

Extracts of each CSF specimen from study patients were inoculated in a 0.2 mL volume into primary rhesus monkey kidney (PRMK) tube cell culture (Diagnostic Hybrids, Athens, Ohio; and Viromed Laboratories, Minneapolis, Minn.) and incubated at 35-37° C. for up to 14 days. Cytopathic effects (CPE) were observed by the formation of large, somewhat irregularly shaped granular cells occurring randomly throughout the culture with cell debris floating in the medium.

### Example 3

### Results

Performance characteristics of the primers and probes used for the detection of enterovirus were determined by comparing the real-time PCR method described herein with standard cell culture. Of 715 CSF specimens, 60 samples were positive by both real-time PCR and cell culture methods (Table 3). Twenty-two specimens and five specimens were exclusively positive by real-time PCR and cell culture methods, respectively. Six hundred twenty-eight specimens were negative by both methods. These comparisons resulted in the following characteristics of the real-time PCR method described herein compared with standard cell culture: sensitivity, 92%; specificity, 97%; positive predictive value, 73% and negative predictive value, 99%. These data demonstrate that the LightCycler detection assay is highly sensitive and specific. The assay's negative predictive value is particularly high, indicating that a negative result truly reflects the absence of enterovirus.

TABLE 3
Comparison of Real-Time PCR and Cell Culture Detection Methods

| | Culture | | |
|---|---|---|---|
| LightCycler | Positive | Negative | Total |
| Positive | 60 | 22* | 82 |
| Negative | 5* | 628 | 633 |
| Total | 65 | 650 | 715 |

*Statistically significant (p = 0.0015)

### Other Embodiments

It is to be understood that while this document has been described in conjunction with the detailed description thereof, the foregoing description is intended to illustrate and not limit the scope of this document, which is defined by the scope of the appended claims. Other aspects, advantages, and modifications are within the scope of the following claims.

What is claimed is:

1. A method for detecting the presence or absence of enterovirus in a biological sample from an individual, said method comprising:
performing at least one cycling step, wherein a cycling step comprises an amplifying step and a hybridizing step, wherein said amplifying step comprises contacting said sample with a pair of enterovirus primers to produce an enterovirus amplification product if an enterovirus nucleic acid molecule is present in said sample, wherein said pair of enterovirus primers comprises a first enterovirus primer and a second enterovirus primer, wherein said first enterovirus primer comprises the sequence 5′-CCG GCC CCT GAA TG-3′ (SEQ ID NO:1), and wherein said second enterovirus primer comprises the sequence 5′-CAC CGG ATG GCC AAT-3′ (SEQ ID NO:2), wherein said hybridizing step comprises contacting said sample with
(a) a pair of enterovirus probes, wherein the members of said pair of enterovirus probes hybridize to said enterovirus amplification product within no more than five nucleotides of each other, wherein a first enterovirus probe of said pair of enterovirus probes is labeled with a donor fluorescent moiety and said second enterovirus probe of said pair of enterovirus probes is labeled with a corresponding acceptor fluorescent moiety, wherein said first enterovirus probe comprises the sequence 5′-GGG CAA CTC TGC AGC GGA ACC GAC-3′ (SEQ ID NO:3), and wherein said second enterovirus probe comprises the sequence 5′-TGG GTG ACC GTG TTT CTT TT-3′ (SEQ ID NO:4); or
(b) one enterovirus probe, wherein said enterovirus probe comprises the sequence 5′-GGG CAA CTC TGC AGC GGA ACC GAC-3′ (SEQ ID NO:3) or the sequence 5′-TGG GTG ACC GTG TTT CTT TT-3′ (SEQ ID NO:4), wherein said enterovirus probe is labeled with a first fluorescent moiety and a second fluorescent moiety, wherein said first and second fluorescent moieties are within no more than 5 nucleotides of each other on said enterovirus probe;
and
detecting the presence or absence of fluorescence resonance energy transfer (FRET) between said donor fluorescent moiety of said first enterovirus probe and said acceptor fluorescent moiety of said second enterovirus probe,
wherein the presence of FRET is indicative of the presence of enterovirus in said sample, and wherein the absence of FRET is indicative of the absence of enterovirus in said sample.

2. The method of claim 1, wherein said donor fluorescent moiety is fluorescein.

3. The method of claim 1, wherein said detecting step comprises exciting said sample at a wavelength absorbed by said donor fluorescent moiety and visualizing and/or measuring the wavelength emitted by said corresponding acceptor fluorescent moiety.

4. The method of claim 1, wherein said detecting comprises quantitating said FRET.

5. The method of claim 1, wherein said detecting step is performed after each cycling step.

6. The method of claim 1, further comprising determining the melting temperature between one or both of said enterovirus probe(s) and said enterovirus amplification product, wherein said melting temperature confirms said presence or said absence of said enterovirus.

7. The method of claim 1, wherein said biological sample is cerebrospinal fluid.

8. The method of claim 1, wherein said amplification employs a polymerase enzyme having 5′ to 3′ exonuclease activity.

9. The method of claim 1, wherein said enterovirus probe comprises a nucleic acid sequence that permits secondary structure formation, wherein said secondary structure formation results in spatial proximity between said first and second fluorescent moiety.

10. A method for detecting the presence or absence of enterovirus in a biological sample from an individual, said method comprising:
performing at least one cycling step, wherein a cycling step comprises an amplifying step and a dye-binding step, wherein said amplifying step comprises contacting said sample with a pair of enterovirus primers to produce an enterovirus amplification product if an enterovirus nucleic acid molecule is present in said sample, wherein said dye-binding step comprises contacting said enterovirus amplification product with a double-stranded DNA binding dye; and
detecting the presence or absence of binding of said double-stranded DNA binding dye into said amplification product,
wherein the presence of binding is indicative of the presence of enterovirus in said sample, and wherein the absence of binding is indicative of the absence of enterovirus in said sample.

11. The method of claim 10, further comprising determining the melting temperature between said enterovirus amplification product and said double-stranded DNA binding dye, wherein said melting temperature confirms said presence or absence of said enterovirus.

12. An oligonucleotide having a sequence selected from the group consisting of 5′-CCG GCC CCT GAA TG-3′ (SEQ ID NO:1); 5′-CAC CGG ATG GCC AAT-3′ (SEQ ID NO:2); 5′-GGG CAA CTC TGC AGC GGA ACC GAC-3′ (SEQ ID NO:3); and 5′-TGG GTG ACC GTG TTT CTT TT-3′ (SEQ ID NO:4).

13. An article of manufacture, comprising:
a pair of enterovirus primers;
a pair of enterovirus probes; and
a donor fluorescent moiety and a corresponding acceptor fluorescent moiety,
wherein said pair of enterovirus primers comprise a first enterovirus primer and a second enterovirus primer, wherein said first enterovirus primer comprises the sequence 5′-CCG GCC CCT GAA TG-3′ (SEQ ID NO:1), wherein said second enterovirus primer comprises the sequence 5′-CAC CGG ATG GCC AAT-3′ (SEQ ID NO:2), and
wherein said pair of enterovirus probes comprises a first enterovirus probe and a second enterovirus probe, wherein said first enterovirus probe comprises the sequence 5′-GGG CAA CTC TGC AGC GGA ACC GAC-3′ (SEQ ID NO:3), and wherein said second enterovirus probe comprises the sequence 5′-TGG GTG ACC GTG TTT CTT TT-3′ (SEQ ID NO:4).

14. The article of manufacture of claim 12, wherein said first enterovirus probe is labeled with said donor fluorescent moiety and wherein said second enterovirus probe is labeled with said corresponding acceptor fluorescent moiety.
